(19) United States
(12) Patent Application Publication
(10) Pub. No.: US 2016/0199497 A1
(43) Pub. Date: Jul. 14, 2016

(54) Cholesterol Ester-Depleting Nanomedicine for Non-toxic Cancer Chemotherapy

(71) Applicant: Purdue Research Foundation, West Lafayette, IN (US)

(72) Inventors: Ji-xin Cheng, West Lafayette, IN (US); Seung Young Lee, West Lafayette, IN (US)

(73) Assignee: PURDUE RESEARCH FOUNDATION, West Lafayette, IN (US)

(21) Appl. No.: 14/850,941

(22) Filed: Sep. 10, 2015

Related U.S. Application Data
(60) Provisional application No. 62/048,495, filed on Sep. 10, 2014.

(51) Int. Cl. A61K 47/42; A61K 31/255

(52) U.S. Cl. CPC A61K 47/42; A61K 31/255

(57) ABSTRACT

The present invention provides a formulation for reducing the hydrophobicity of ACAT-1 inhibitors. Methods for using the formulation of the present invention are also provided.

This invention was made with government support under CA129287 awarded by the National Institutes of Health. The government has certain rights in the invention.

### TECHNICAL FIELD

The present disclosure generally relates to a composition and a method of treating cancer, and in particular to treating cancer using a cholesterol esterification inhibitor.

### BACKGROUND

Metabolic pathways of cancers have recently been recognized as novel targets for selective chemotherapy because proliferating cancer cells exhibit remarkably distinct metabolic requirements compared to most non-cancerous, differentiated cells. In particular, altered lipid metabolism including cholesterol is an emerging target for aggressive cancers. Cholesterol is an essential component for the cell membrane and for signaling molecule synthesis; cancer cells produce and use larger amounts of cholesterol for their development and growth. Lipid metabolic reprogramming allows cancer cells not only to obtain substantial cholesterol through low-density lipoprotein (LDL) uptake and de-novo biosynthesis but also to accumulate cholesterol into their intracellular lipid reservoir, lipid droplets (LDs), after converting cholesterol to cholesteryl esters (CEs). Because high intracellular cholesterol levels induce cytotoxicity, cholesterol esterification by acyl-CoA cholesterol acyltransferase-1 (ACAT-1) is a key process in cancer cells.

ACAT-1 inhibitors are known to block the uptake and storage of cholesteryl esters in LDs resulting in free CEs which cause stress and cell death. The problem with using currently available ACAT-1 inhibitors to inhibit cancer growth is that the drugs are not soluble and thus are low in bioavailability. Therefore a new formulation is needed to make use of the cancer inhibition effects of these ACAT-1 inhibitors.

### SUMMARY OF THE INVENTION

The present invention provides a formulation comprising an original ACAT-1 inhibitor and human serum albumin, wherein the combination of the original ACAT-1 inhibitor and human serum albumin decreases the hydrophobicity of the original ACAT-1 inhibitor. The formulation may increase the solubility of the ACAT-1 inhibitor by as much as 40-fold or greater. Moreover, the formulation may have an ACAT-1 loading of from about 10 wt % to about 30 wt % or from about 15 wt % to about 25 wt %, with respect to human serum albumin. The formulation of claim 1 wherein the original ACAT-1 inhibitor has a loading of from about 15 wt % to about 25 wt % with respect to human serum albumin. In an illustrative aspect, the original ACAT-1 inhibitor may be avasimibe.

The present invention also provides a method of treating a mammal having a disease or condition that would benefit from inhibiting ACAT-1 comprising administering a therapeutically effective amount of a formulation comprising an original ACAT-1 inhibitor and human serum albumin, wherein the combination of the original ACAT-1 inhibitor and human serum albumin decreases the hydrophobicity of the original ACAT-1 inhibitor. The formulation may be administered by systemic or oral administration.

### BRIEF DESCRIPTION OF THE FIGURES

FIG. 1A is a graph showing the CD spectra of human serum albumin (HSA) at different avasimibe:HSA molar ratios. Inserted into FIG. 1A shows a plot of CD intensity at 208 nm as a function of avasimibe/HSA;

FIG. 1B is a graph quantifying the solubility of avasimibe and new formulation ACAT-1 inhibitor, avasimin;

FIG. 2A is a bar graph that quantifies the area (%) of a lipid droplet (LD) in single cells having been treated with PBS (negative control) or avasimin;

FIG. 2B is a graph which quantifies the molar percentage (%) of CE in LDs based on the Raman spectra;

FIG. 3A is a bar graph illustrating the cell viability percentages of the cancer cell lines and non-cancerous cell lines with increasing avasimin concentration;

FIG. 3B is a bar graph showing the fold increase of free cholesterol in each cancer cell line comparing PBS to avasimin, wherein the amount of free cholesterol is normalized by the PBS treated cells;

FIG. 3C is a bar graph quantifying the percentage of apoptotic cells;

FIG. 4A is a graph showing the amount of bioavailable avasimibe in the unformulated, oral dosage or in the new formulated avasimin which is intravenously injected into an animal model;

FIG. 4B is a bar graph quantifying the concentration of either avasimibe or avasimin (comprising 15 mg/kg of avasimibe) in various tissues and organs in the murine animal model.

FIG. 5A is a graph showing tumor volume produced from PC3 cells following treatment of avasimin or PBS over a period of time and the survival rate for each group;

FIG. 5B is a graph showing tumor volume produced from HCT116 cells following treatment of avasimin or PBS over a period of time and the survival rate for each group.

FIG. 5C is a bar graph illustrating the CE percentage (%) in LDs in the tumor tissues based on the Raman spectra;

FIG. 5D is a bar graph depicting the fold increase of free cholesterol when the tumor is treated with PBS or avasimin, and the data is normalized by the PBS treated group;

FIG. 5E is a bar graph quantifying the number of apoptotic cells in the tumor tissues;

FIG. 6A is bar graphs quantifying the complete blood count and serum analyses of a murine animal model after intravenous injection of avasimin or PBS; and

FIG. 6B is a graph quantifying the CE percentage (%) in adrenal gland lipid droplets in the murine adrenal gland tissue, based on Raman spectra.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

By “original ACAT-1 inhibitor” refers to a compound designed to inhibit the activity of acetyl-CoA acetyltransferase 1 (ACAT-1) activity in a cell, which comprises a high purity of at least 95% ACAT-1 inhibitor compound and does not include human serum albumin (HSA).

By “effective amount” refers to an amount of pharmaceutical composition, such as avasimin, that is able to reduce, inhibit proliferation, or treat a disease such as cancer. The effective amount will vary depending on several factors associated with the mammal and the disease itself, such as tumor size. The effective amount is able to be determined by one of skill in the art.

Broadly, the present invention provides a formulation comprising an original ACAT-1 inhibitor and human serum albumin (HAS), wherein the formulation increases the bioavailability and solubility of the original ACAT-1 inhibitor by reducing the hydrophobicity. The formulation may be referenced as avasimin herein, particularly in the examples below. The ACAT-1 inhibitor binds to the HSA in a specified ratio, increasing the solubility of the ACAT-1 inhibitor. An advantage of the formulation of the present invention is that, while increasing the solubility of the ACAT-1 inhibitor about 40-fold, the formulation does not inhibit the bioavailability of the ACAT-1 inhibitor which may still inhibit ACAT-1 in a cell and/or an organism. Methods are also provided for administering the formulation of the present invention to a mammal in order to treat a disease or condition that will benefit from the inhibition of ACAT-1.

In one aspect of the present invention, the original ACAT-1 inhibitor is mixed with HSA. By way of non-limiting example, the ACAT-1 inhibitor is mixed with the HSA in solution. The amount, or loading of the ACAT-1 inhibitor to HSA may be from about 1 wt % to about 35 wt %, or from about 10 wt % to about 30 wt %. In an exemplary aspect of the present invention, the loading of the ACAT-1 inhibitor to HSA may be from about 15 wt % to about 25 wt %. The optimal loading may be determined by the solubility of the formulation of the present invention. It will be appreciated that increased loading of the ACAT-1 inhibitor will produce a formulation having a greater efficacy when administered to a patient. However, this must be weighed against solubility. The solubility advantaged gained by the formulation of the present invention compared to the ACAT-1 inhibitor alone may begin to decrease as the hydrophobicity of the ACAT-1 inhibitor increases in relation to the hydrophilicity of the HSA.

In an alternate aspect of the present invention, the amount of the ACAT-1 inhibitor to the HSA may be expressed as a molar ratio. In one aspect of the present invention the ratio of the original ACAT-1 inhibitor to HSA may 1:99, 2:98, 3:97, 4:96, 5:95, 6:94, 7:93, 8:92, 9:91, 10:90, 15:85, 20:80, 25:75, 30:70, 35:65, 40:60, 45:55, 50:50, 55:45, 60:40, 65:35, 70:30, 75:25, 80:20, 85:15, 90:10, or 95:5. Alternatively, the ratio of the original ACAT-1 inhibitor to HSA may be expressed as a molar ratio, wherein the molar ratio may be from about 1:5 to about 1:50 or from about 1:10 to about 1:35.

In an exemplary aspect of the present invention, the original ACAT-1 inhibitor may be avasimibe, a potent ACAT-1 inhibitor. Although a potent ACAT-1 inhibitor, avasimibe has the disadvantage of being hydrophobic and therefor limited in the therapeutic amount that can be delivered to a patient.

In one aspect of the present invention methods are provided for treating a patient having a disease or condition that may benefit from inhibition of ACAT-1. Non-limiting examples of such diseases may be cancers, such as prostate, pancreatic, lung and colon cancer. The formulation of the present invention allows for a therapeutic dose of the ACAT-1 inhibitor to be administered to a patient. A patient is considered to be any mammal or animal that requires treatment. Examples may be, but not limited to, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like.

In one aspect the formulation of the present invention may be a liquid, a suspension or a solid, such as in capsule or tablet form. In another aspect of the present invention the formulation may be administered systemically, orally, peritoneally, or any advantageous manner as determined by a clinician. In other aspects, avasimin may be administered to a mammal in liquid form. This liquid form may be ingested, inhaled, injected by needle, provided intravenously, catheter, port, or absorbed through the organ membrane of the mammal. The amount of a single dose of ACAT-1 inhibitor included in the formulation of the present invention may range from 10 μg/kg up to 1000 mg/kg, or 10 ng/mL up to 1000 mg/mL, or 10 μL/mL up to 1000 μL/mL. It will be appreciated that those skilled in the art of treating diseases such as cancer can best determine the amount to be administer based on the disease, the extent of the disease, the condition of the patient, the size of the patient and other relevant parameters.

In another aspect of the present invention, the formulation disclosed herein may be administered to a patient in combination with another therapy. This is particularly advantageous in treating diseases such as cancer where the patient may develop a resistance to the drug being administered. In an illustrative aspect of the present invention, when treating a patient for cancer the formulation disclosed herein may be combined with traditional therapies such abraxane, fluorouracil, everolimus, erloinib HCl, gemcitabine, mitomycin C, paclitaxel, and sunitinib malate as well as other combination therapies such as folfirinox, or gemcitabine-cisplatin. It will be appreciated that this list of compounds is provided for illustrative purposes and is not meant to be limiting. In another aspect of the present invention, it has been shown that avasimibe has a synergistic effect when given in combination with gemcitabine to mice with tumors. This synergistic effect is observed at all ratios of avasimibe and gemcitabine, from a molar ratio of about 5:1 to a molar ratio of about 15:1 avasimibe to gemcitabine.

The following are non-limiting examples. It will be understood that some of these techniques are well known in the art, as well as alternatives, and thus should not be limited by only these methods if others are reasonably known.

### Example 1

### Material and Methods

Avasimibe solution (0-0.02 mg in 10 μl ethanol) was added to HSA (human serum albumin) solution (0.08 mg in 990 μl deionized water). The final molar concentrations of avasimibe and HSA were 0-40 μM and 1.2 μM, respectively. After incubation for 1 h at room temperature, circular dichroism (CD) spectra of the avasimibe-HSA solutions were measured using a JASCO J-810 spectropolarimeter (Tokyo, Japan) at 25° C. under a constant nitrogen flow. A quartz cell of 1 mm path length was used for spectrum measurement (190-260 nm). The spectra were collected with a data pitch of 1 nm, a scan speed of 10 nm per minute, and bandwidth of 1 nm. Each spectrum was the average of three scans.

To find the binding site of avasimibe on HSA, fluorescence emission spectrum change of tryptophan (Trp-214) in HSA by adding avasimibe was measured using a fluorescence spectrometer (SpectraMax M5, Molecular Devices, CA) with excitation at 280 nm and emission scanning from 300 nm to 450 nm at 25° C.

To determine the avasimibe binding to the drug binding site I on HSA, a competitive binding assay using warfarin was performed. Warfarin solution (0.004 mg in 10 μl ethanol) was mixed with HSA solution (0.08 mg in 980 μl deionized water), and the solution was incubated for 1 h at room temperature. Avasimibe (0.01 mg in 10 μl ethanol) was added to the warfarin-HSA solution. The warfarin, warfarin-HSA, warfarin-HSA+ avasimibe solutions had the same mixed solvent composition of ethanol (20 μl) and water (980 μl). Fluorescence emission spectra of warfarin in the solutions were measured with excitation at 335 nm and emission scanning from 355 nm to 550 nm at 25° C.

Avasimibe was prepared using a modified nanoencapsulation method with HSA as a stabilizer. In brief, avasimibe solution (1-2 mg in 1 ml ethanol) was added to HSA solution (4 mg in 4 ml PBS), and then the solution was incubated at 4° C. for 8 h. Non-trapped avasimibe and ethanol were removed by dialysis (MWCO 12-14 kDa) against excess deionized water for 1 day and centrifugation (×1600 g, 10 min). The resulting solutions were lyophilized.

Loading amount of avasimibe into avasimin was determined by UV absorption of avasimibe at 275 nm after HSA removal by acetone precipitation twice. The size distribution of avasimin (at 1 mg/ml PBS) was characterized using dynamic light scattering (DLS) (90Plus, Brookhaven Instruments Co., NY) at 633 nm at 25° C. The zeta-potential of avasimin (at 1 mg/ml PBS) was also measured using a zeta-potential analyzer (ZetaPlus, Brookhaven Instruments Co., NY). The turbidities of avasimibe and avasimin solutions containing different concentrations of avasimibe (0-2 mg/ml PBS) were measured by UV absorption at 430 nm. The morphologies of avasimibe, avasimin, and HSA in PBS or distilled water were observed using an FV1000 confocal system (Olympus, Tokyo, Japan) and transmission electron microscopy (TEM) (CM200 electron microscope, Philips, Oreg.).

All protocols for this animal study were approved by the Purdue Animal Care and Use Committee. PC3 or HCT116 cancer cells (2×10̂6 cells) were mixed with an equal volume of Matrigel (BD Bioscience) and inoculated subcutaneously into the right flank of 6-week-old male athymic nude mice (Harlan Laboratories).

To compare the blood-bioavailability of avasimibe for oral and intravenous administrations, avasimibe solution in PBS (15 mg/kg) was orally administered to nude mice without tumors (n=3) using a plastic feeding tube (20 GA×38 mm). Avasimin solution in PBS (75 mg/kg, containing 15 mg/kg avasimibe) was intravenously administered to nude mice without tumor (n=3) through the tail vein. Blood samples (2 μl) were collected from the dorsal pedal vein at different time points post administration. The blood sample was mixed with K3-EDTA (0.2 μl, an anticoagulation agent), warfarin solution (5 ng in 1 μl deionized water, an internal standard), and DTT (40 μg in 0.5 μl PBS, a reducing agent). To extract avasimibe, acetone (6 μl) was added to the blood solution, vortexed, and then the solution was centrifuged (×5000 g, 5 min). The supernatant (6 μl) was collected and dried under vacuum. The sample was stored at −20° C., and dissolved in 50 μl of methanol just before analysis. A linear standard curve of avasimibe in blood ranging of 0.01-100 μg/ml was created using the same extraction method described above. avasimibe concentration was determined using a liquid chromatography-mass spectroscopy (LC-MS) (Agilent 1200 HPLC-Agilent 6460 QQQ mass spec). A Zorbax SB-C18 2.1×50 mm, 1.8 μm column (Agilent) was used. The mobile phase consisted of water with 5 mM ammonium acetate (Buffer A) and 90% acetonitrile with 5 mM ammonium acetate (Buffer B) was delivered at a flow rate of 0.3 ml/min. The sample injection volume was 10 μl. The retention times of the analyte (avasimibe) and the internal standard (warfarin) were 7.9 min and 6.6 min, respectively. To detect avasimibe, the mass spectrometer was operated in the multiple-reaction monitoring (MRM) mode, and was set to select the precursor-product ion transitions of m/z 500.1→177.0 and m/z 307.0→161.1 for avasimibe and warfarin, respectively.

The dataset was fit to a two-compartment pharmacokinetic model for avasimibe profile

(R²=0.99): y=A₁e^{(-x/t1)}+A₂e^{(-x/t2)}+y₀

Based on this model, pharmacokinetic values for avasimibe delivered by intravenous administration of avasimin were determined including the initial volume of distribution,

V₀: V₀=Dose/C₀(initial concentration)

The plasma clearance, Cl_{Plasma}: Cl_{Plasma}=V₀×k (elimination rate constant)
The area under curve, AUC: AUC=Dose/Cl_{Plasma}
The life-time for one-compartment model, t_{1/2}:

t_{1/2}=0.693/k

The terminal half-time for two-compartment model, t_{1/2}, term:

t_{1/2,term}=2 Ln [2]/[k+kₚₜ+kₜₚ−((k+kₚₜ+Kₜₚ)²−4k kₜₚ)^{0.5}]

And the distribution half-time for two-compartment model, t_{1/2, dist}:

t_{1/2,dist}=Ln [2][k+kₚₜ+kₜₚ−((k+kₚₜ+Kₜₚ)²−4k kₜₚ)^{0.5}]/[2k kₜₚ]

The terminal and distribution half-times for two-compartment model were measured using the equations above, described in a previous report.

To measure the bioavailability of avasimibe in tissues, avasimin solution in PBS (75 mg/kg, containing 15 mg/kg avasimibe) was intravenously administered to PC3 tumor xenograft nude mice (n=3) through the tail vein. avasimibe solution in PBS (15 mg/kg) was also orally administered to PC3 tumor xenograft nude mice (n=3). At 2 hr-post injection, the mice were sacrificed, and the tissues and urine were harvested and weighed. After adding PBS (100 μl per 100 μg tissue), the tissues were homogenized using a bead based homogenizer (Precellys®24, bertin technologies). Avasimibe was extracted from the homogenized tissue solution according to the same procedure used in avasimibe extraction from blood sample. Avasimibe in the tissue, urine, and feces specimens was quantified by LC-MS using the same conditions as the blood-bioavailability study. The amounts of avasimibe were quantitatively determined based on the calibration curve of avasimibe in blood.

Anti-tumor effect of avasimin was evaluated by measuring the tumor volume and survival length. After reaching a tumor volume of 30-40 mm³, PC3 and HCT116 tumor xenograft mice were randomized into two groups (n=8 per PBS or avasimin group). The mice received avasimin (75 mg/kg, containing 15 mg/kg avasimibe) and PBS by intravenous injection daily for the first 5 days, followed by intravenous injection once every 4 days. The body weight of the mice was monitored during the treatment. Tumor volume was measured as follows:

V=(a×b²)/2

where a and b represent the major and minor axes of a tumor, respectively. The lengths of the axes were measured using a caliper. When tumor volume reached over 10% of body weight, mice were sacrificed and tumors were harvested for tissue analysis.

TUNEL and DAPI staining. The tumor tissues were fixed in 10% neutral buffered formalin for at least 48 h, embedded into paraffin, sectioned at 5 μm thickness. The sections were stained with terminal deoxynucleotidyl transferase (TdT) and 4,6-diamidino-2-phenylindole (DAPI) according to the protocol of in situ cell death detection kit (Roche).

In vivo safety evaluation. Nude mice without tumors received avasimin (75 mg/kg, containing 15 mg/kg avasimibe) and PBS by intravenous injection once every 4 days for 16 days (four injections in total). The mice were sacrificed, blood and organs were collected. Complete blood count (CBC) and serum analyses were performed by Antech Diagnostics. The organs were fixed in 10% neutral buffered formalin for at least 48 h, embedded into paraffin. Sections of 5 μm thickness were stained with hematoxylin and eosin in Purdue University Histopathology Lab. The slides were then examined on a Nikon microscope equipped with a charge-coupled device camera.

Results

Water-Soluble Avasimibe Formulation:

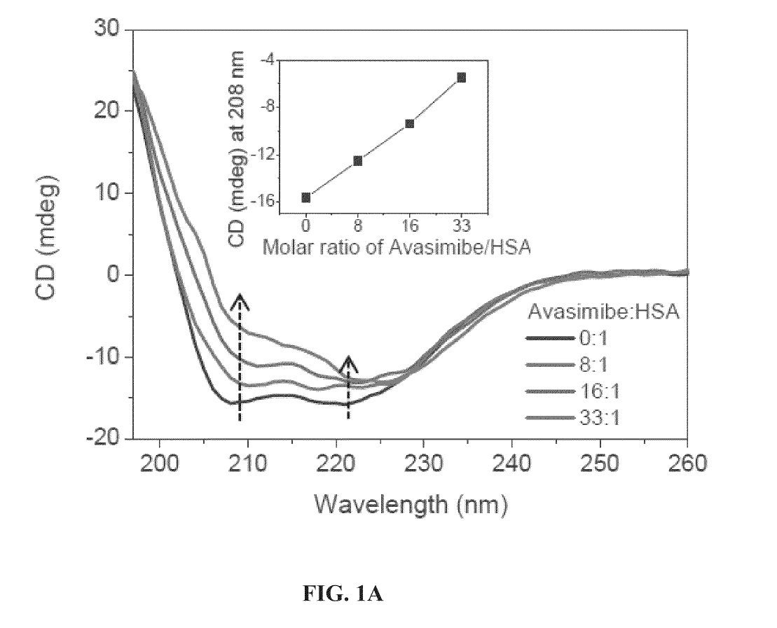

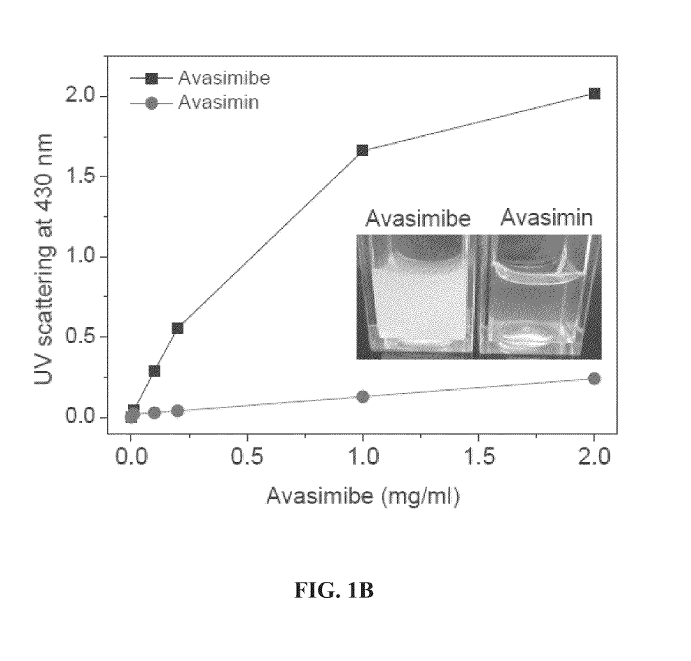

To develop an injectable and non-toxic avasimibe formulation, human serum albumin (HSA) was utilized due to its high biocompatibility and binding capability for hydrophobic molecules. The binding property of avasimibe to HSA was first determined by circular dichroism (CD) and fluorescence spectroscopic analyses. As shown in FIG. 1A, the CD spectra of HSA exhibited two negative bands in the ultraviolet region at 208 nm and 222 nm, which is characteristic of the α-helical structure of protein. By adding avasimibe, however, the intensities of the negative CD bands decreased notably, until the avasimibe:HSA molar ratio reached 33:1. The peak intensity change at 208 nm as a function of the avasimibe:HSA molar ratio was clearly shown (Insert in FIG. 1A). This decrease in the α-helical content indicates the binding of avasimibe to HSA. Also, fluorescence quenching of the single tryptophan residue, Trp-214, demonstrated that the subdomain IIA of HSA is the avasimibe binding site. In addition, the magnitude of the intensity change suggests that at an avasimibe:HSA molar ratio of 16:1, the binding site is fully occupied by avasimibe. To further confirm the avasimibe binding site, warfarin was employed as a site I marker. The binding site of warfarin to HSA was designated as site I, located in subdomain IIA near Trp-214. Warfarin showed a fluorescence emission at 390 nm on excitation at 335 nm, and the addition of HSA (at warfarin:HSA molar ratio of 10:1) increased the fluorescence intensity due to warfarin binding to site I in the protein. However, the addition of avasimibe (at avasimibe:HSA molar ratio of 16:1) caused the fluorescence of warfarin to return to its original intensity, indicating a complete replacement of warfarin by avasimibe at the primary binding site of HSA. This data suggests that the binding affinity of avasimibe to HSA is higher than that of warfarin (1.89×10⁵ M⁻¹). Collectively, these results show that avasimibe strongly binds to HSA and that the binding site is located in subdomain IIA.

Based on the above results, a water-soluble avasimibe formulation was prepared, referred to as avasimin, using a modified nanoencapsulation method. The optimal loading amount of avasimibe was 20 wt % determined by a turbidity test of avasimin with different drug concentrations. The water-solubility of avasimibe and avasimin in PBS was compared using UV scattering at 430 nm (FIG. 1B). The higher the UV scattering intensity, the more turbid the solution is. The turbidity measurement confirmed that the avasimin formulation increased the water-solubility of avasimibe by up to 40 times. The microscopic image of avasimibe solution showed undissolved and needle-shaped crystals of the drug, whereas the avasimin solution was clear. The TEM image permitted the visualization of the nano-sized and spherical morphology of avasimin. High-magnification TEM revealed HSA coating on the surface of avasimin. In addition, the mean diameter and surface charge of avasimin in PBS were measured to be 169 nm and 0.83 mV, respectively. Together, these data demonstrate that the avasimin formulation effectively improved the water-solubility of the drug, thus allowing intravenous administration.

CE Depletion and Cholesterol Cytotoxicity in Cancer Cells Induced by Avasimin:

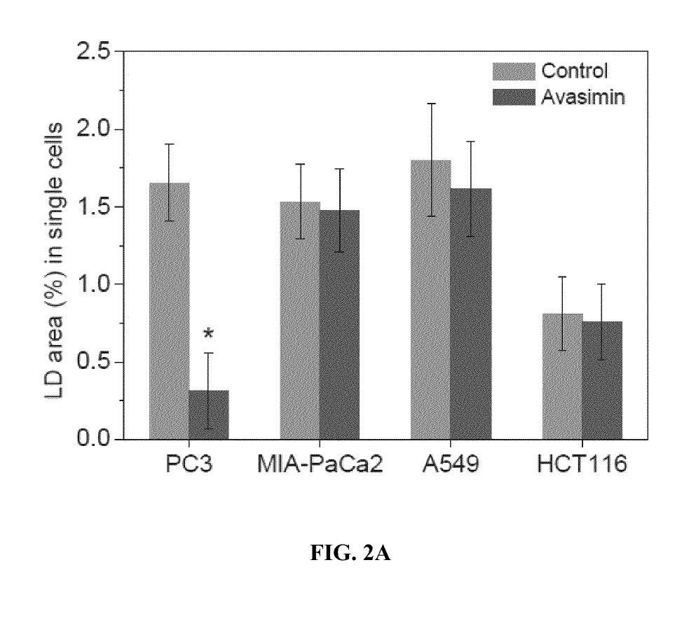

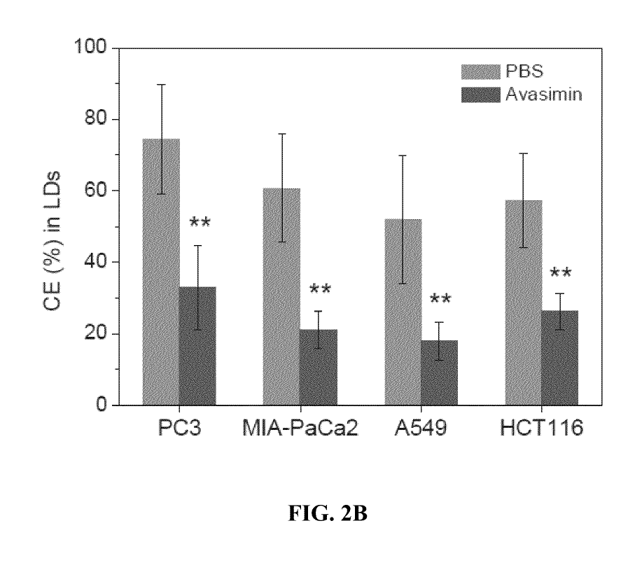

The accumulation of cholesteryl esters (CEs) in lipid droplets (LDs) was determined in a panel of aggressive cancer cells and normal cells. By tuning the laser-beating frequency to be resonant with the C-H stretching vibration, substantial stimulated Raman loss (SRL) signals arose from intracellular LDs. The SRL images showed significant amounts of LDs in various human cancer cells (PC3: Prostate cancer, MIA-PaCa2: Pancreatic cancer, A549: Lung cancer, HCT116: Colon cancer), whereas negligible amounts of LDs in normal cells (hVSMC: Human vascular smooth muscle cells, BR5: Dermal fibroblast) were observed. Notably, avasimin treatment reduced the amount of LDs only in PC3 cells, but not in other cancer cells (FIG. 2A). Next, the composition in LDs was investigated using confocal Raman spectral analysis. Raman spectra from different LDs in the cancer cells were measured, and then the spectra were averaged to form a single spectrum. The Raman spectrum of an LD in cancer cells exhibited the characteristic band for cholesterol ring vibration at 702 cm⁻¹. The band at 702 cm⁻¹ disappeared with avasimin treatment. To determine the CE molar percentage (%) in LD, the height ratio (I₇₀₂/I₁₄₄₂) of the 702 cm⁻¹ peak to the 1442 cm⁻¹ peak (CH₂ bending) in the Raman spectra was calculated, and applied to the linear CE calibration curve obtained by the Raman spectra of CE/triacylglycerol (TG) emulsions with different CE/TG molar ratios. It was found that CEs predominated in LDs for all cancer cell lines (PC3: 74±15%, MIA-PaCa2: 61±15%, A549: 52±18%, HCT116: 57±13%) (FIG. 2B). Avasimin treatment significantly reduced the CE level in LDs for all cancer cells. This CE depletion induced by avasimin also was confirmed using mass spectrometry measurement of CEs in PC3 and MIA-PaCa2 cells.

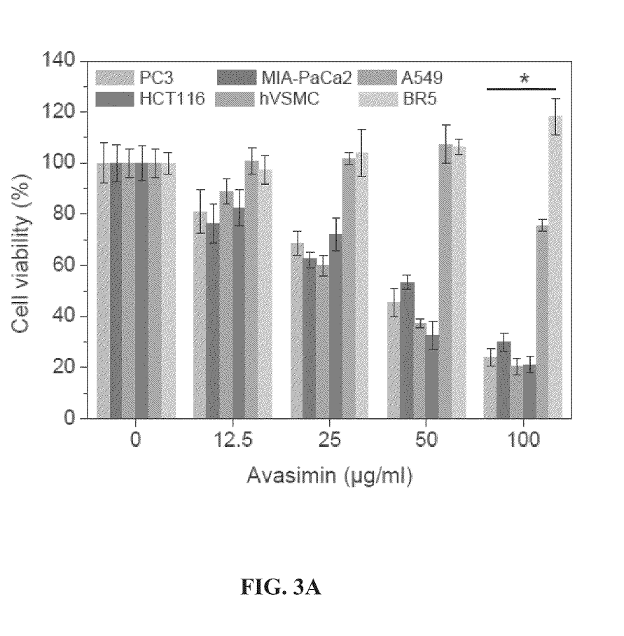

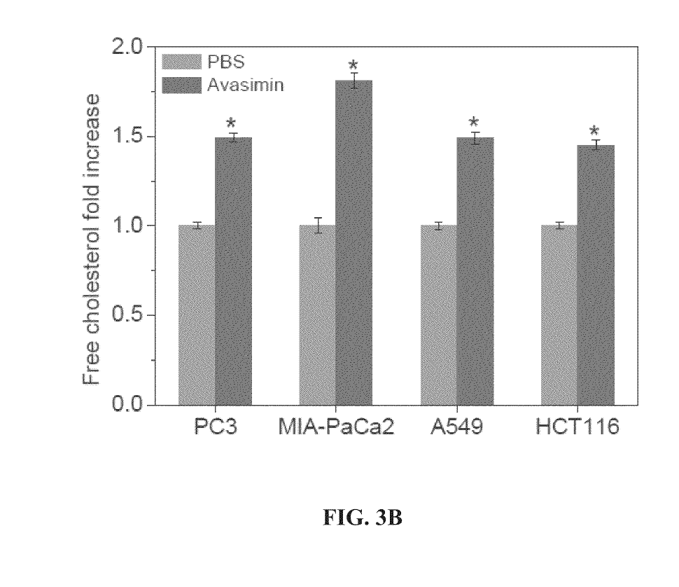

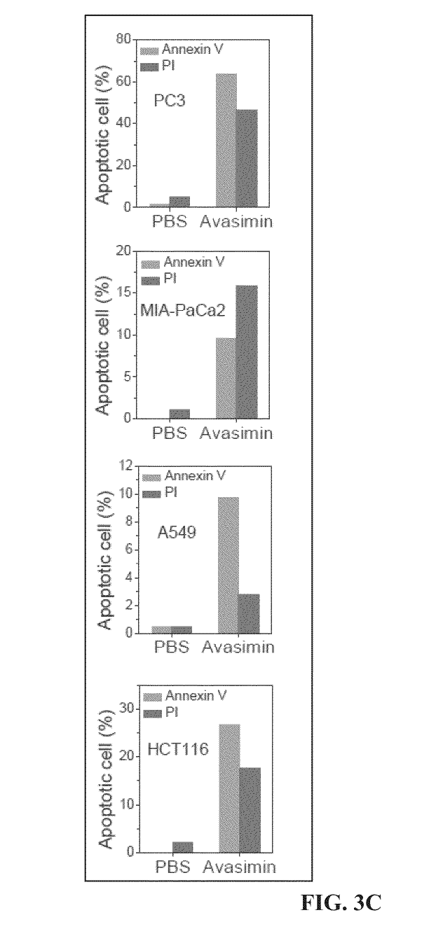

The anti-cancer effect of avasimin was then investigated through cell viability measurement. The treatment of cancer and normal cells with avasimin at different concentrations selectively decreased the viability for cancer cells (FIG. 3A). The half maximal inhibitory concentrations (IC₅₀) of avasimin for the cancer cells were significantly smaller than those for normal cells. It was confirmed that this anti-cancer effect was not caused by HSA. Furthermore, by comparison with non-selective cytotoxic effects of conventional chemotherapeutics such as cisplatin and gemcitabine, the selective inhibitory effect of avasimin on cancer proliferation was clearly proved. To elucidate the inhibitory mechanism, intracellular free cholesterol levels after avasimin treatment were investigated. As shown in FIG. 3B, avasimin increased the amount of free cholesterol in the cancer cells by more than 50%. In accordance, a significant number of early and late apoptotic cells were observed in the avasimin-treated group (FIG. 3C). This apoptosis is likely induced by the high cholesterol level, which stiffens the endoplasmic reticulum (ER) membrane. Collectively, these data show that avasimin selectively suppressed cancer proliferation by free cholesterol-mediated cytotoxicity.

Blood-Residence Time and Tumor Bioavailability of Avasimibe Following Intravenous Administration of Avasimin:

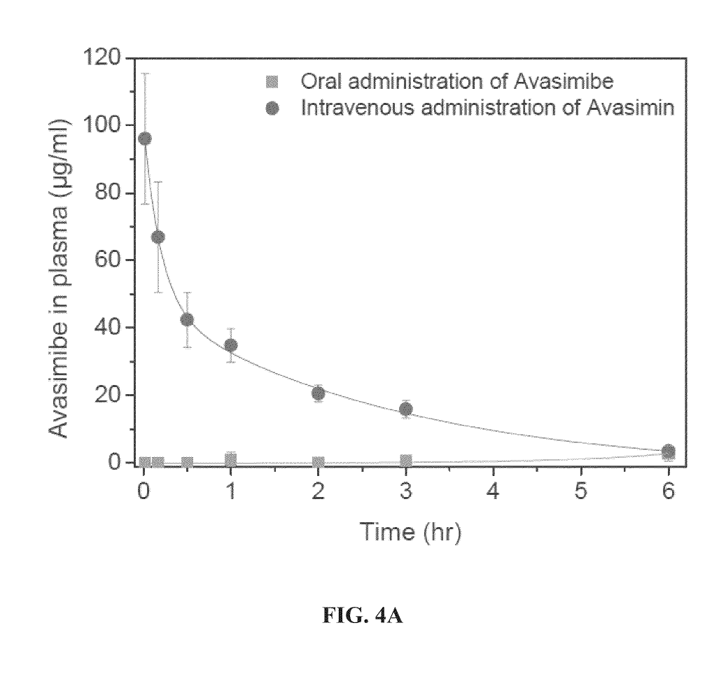

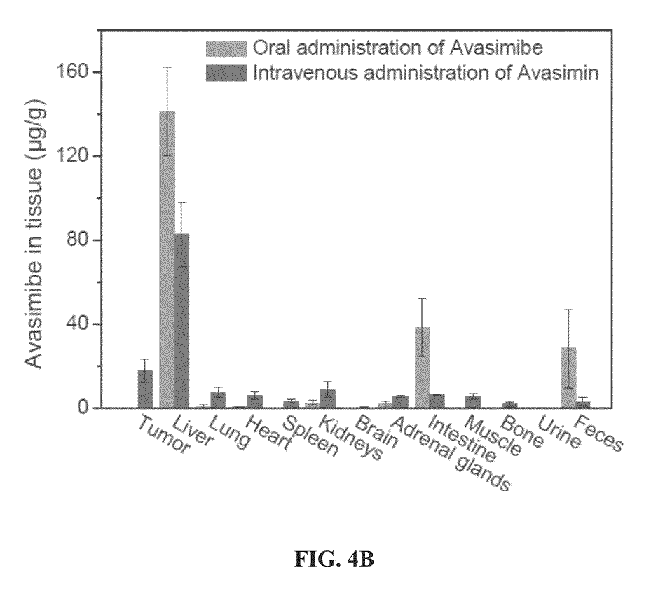

To measure the bioavailability of avasimibe in blood, avasimin (75 mg/kg, containing 15 mg/kg avasimibe) was administered to mice by tail vein injection. Avasimibe (15 mg/kg) was also orally administered to mice as a control. By using liquid chromatography-mass spectroscopy (LC-MS), the plasma avasimibe concentration was measured as a function of time post-injection. A two-compartment pharmacokinetic model was used to fit the plasma concentration profiles (FIG. 4A). The area under curve (AUC) of avasimibe for the intravenous administration of avasimin was 136.36 μg·hr/ml, which was significantly larger than 3.25 μg·hr/ml for the oral administration. The distribution half-life and terminal half-life of avasimibe by intravenous administration of avasimin were 0.14 hr and 1.97 hr, while the half-life of avasimibe by oral administration could not be calculated due to the low plasma concentration. These results show that avasimin effectively increased the blood-bioavailability of avasimibe.

Next, tissue distribution of avasimibe after intravenous administration of avasimin (75 mg/kg, containing 15 mg/kg avasimibe) and oral administration of avasimibe (15 mg/kg) to PC3 tumor-bearing mice was characterized. At 2 hr post administration, the concentrations of avasimibe in tissues, urine and feces were determined using LC-MS (FIG. 4B). The tumor-bioavailability of avasimibe for intravenous administration of avasimin was 17.8 μg/g (34 μM, tumor density: 1.03 g/cm³), which is 2-fold higher than the IC₅₀ of the drug for PC3 cells (17 μM). In contrast, avasimibe was not detectable in the tumor tissue after oral administration. Orally administered drug is known to be absorbed by the gastrointestinal tract, and then carried to the liver via the hepatic portal vein. Accordingly, most avasimibe was found in the liver tissue after the oral administration (FIG. 4B). In the oral administration group, a considerable amount of avasimibe was excreted through the feces. These results correspond to the low blood-bioavailability of avasimibe for the oral administration. Together, these results show that intravenous administration of avasimin significantly increased the drug concentration in the tumor, exceeding the IC₅₀ value.

Anti-Tumor Activity of Avasimin:

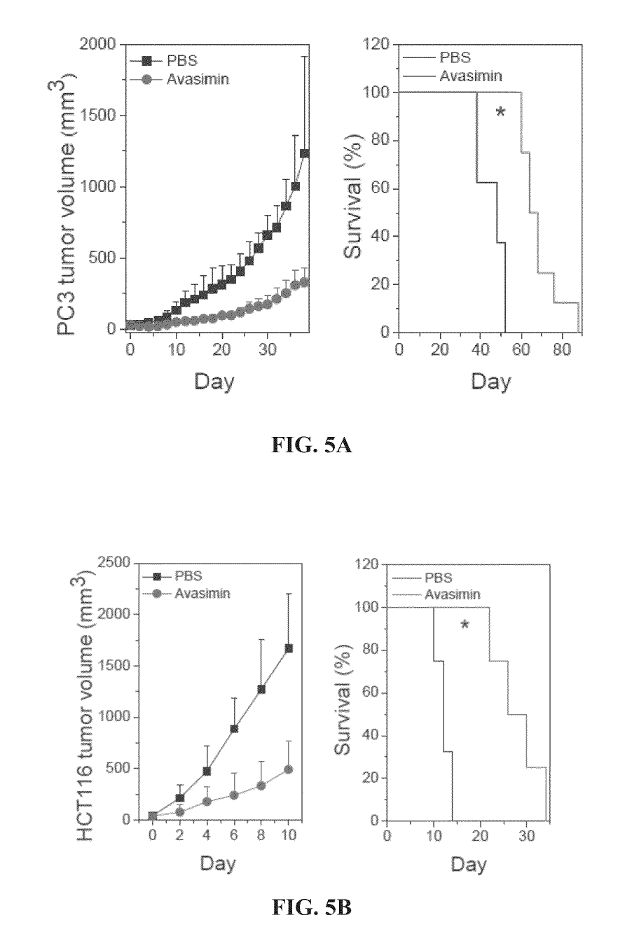

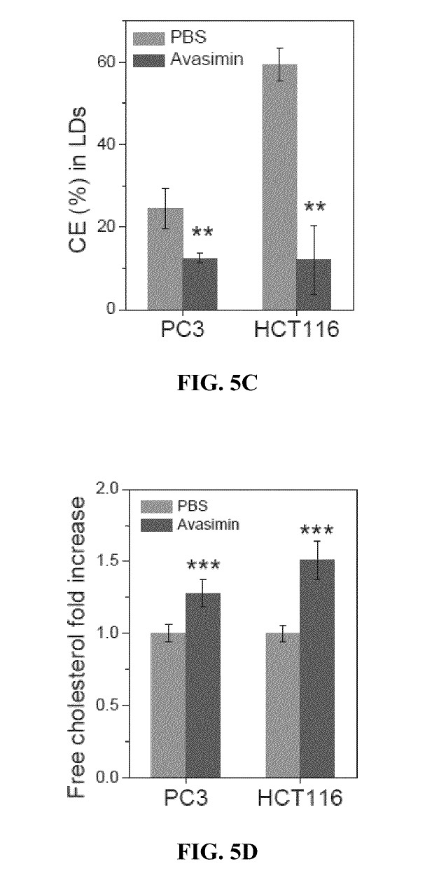

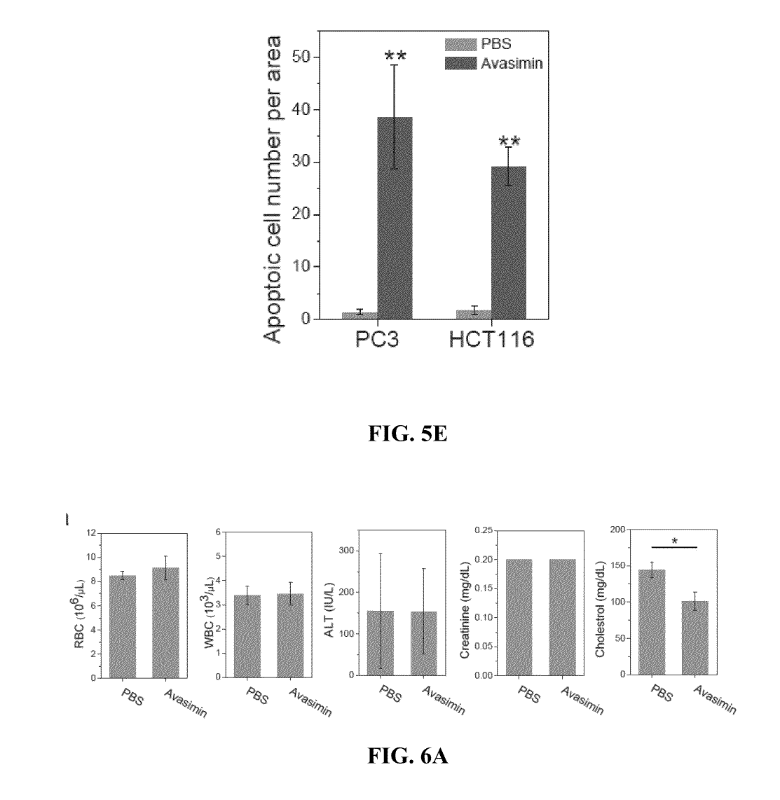

The inhibitory activity of avasimin against subcutaneously xenografted PC3 and HCT116 tumors was evaluated. Avasimin (75 mg/kg, containing 15 mg/kg avasimibe) and PBS were intravenously administered daily for the first 5 days, followed by intravenous injection once every 4 days. In spite of the difference in growth profiles of PC3 and HCT116 tumors, avasimin notably inhibited both PC3 and HCT116 tumor growth, compared to the control groups that received PBS treatment (FIGS. 5A and 5B). Also, the avasimin treatment significantly extended the length of survival time in both tumor models (*P<0.005, by log-rank test). During the treatment, no obvious body weight change was observed.

The pharmacodynamics in both tumor models was further studied. SRL imaging showed a large amount of lipid accumulation in the tumor tissues. In particular, avasimin treatment distinctly reduced the amount of LDs only for the PC3 tumors, not for the HCT116 tumors. Raman spectra from LDs in the tumor tissues demonstrated the cholesterol ring vibration band at 702 cm⁻¹. The CE level (59±14%) in LDs of HCT116 tumor tissue was similar to in vitro cultured HCT116 cells (57±13%), whereas the CE level (25±5%) in LDs of PC3 tumor tissue was smaller than that of cultured PC3 cells (74±15%) (FIG. 5C). This suggests that different environmental conditions can alter lipid accumulation pathways in cancer cells. After repeated avasimin treatment, however, CE level (%) for both PC3 and HCT116 tumors were significantly reduced. The increase (more than 30%) in free cholesterol levels for the tumor treated with avasimin was also determined (FIG. 5D). In addition, TUNEL staining showed a significant number of apoptotic cells in the tumors treated with avasimin, compared to the tumors treated with PBS (FIG. 5E). Collectively, the data demonstrate the potency of avasimin as a promising chemotherapeutic agent for various tumors.

In Vivo Safety Evaluation:

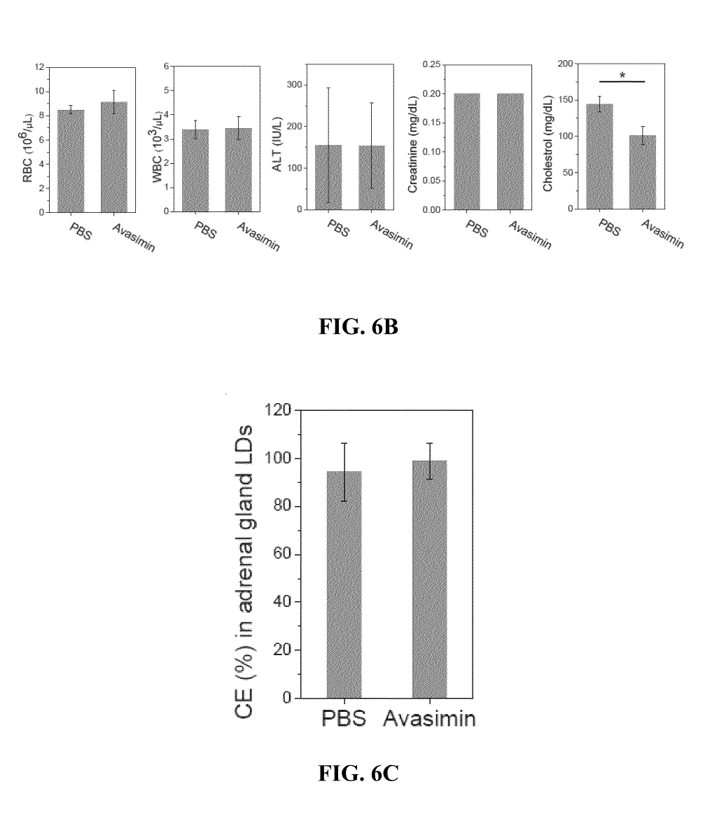

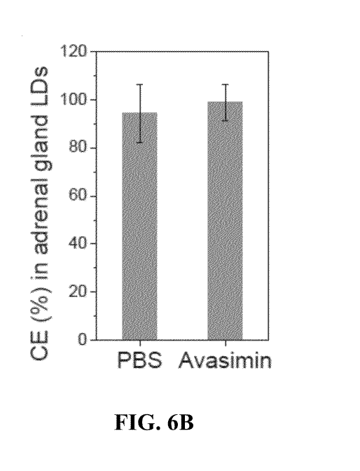

In vivo toxicity of avasimin was evaluated through hematological and histological analyses. Avasimin (75 mg/kg, containing 15 mg/kg avasimibe) was intravenously injected once every 4 days for 16 days (four injections in total). As a control group, PBS was administered in the same manner. The results of hematology and serum analyses between the avasimin-treated and PBS-treated groups were not significantly different, except for cholesterol levels (FIG. 6A). The decrease in the blood cholesterol level in the avasimin-treated group was attributed to the reduction of very low density lipoprotein (VLDL) production in the liver mediated by ACAT-1 inhibition. On the other hand, the levels of creatinine and alanine aminotransferase (ALT) for the avasimin-treated group were the same as those of the PBS-treated group, indicating no damage to the kidneys and the liver. The morphology of vital organs was assessed using H&E staining, and no morphological difference was observed between the groups treated with avasimin and PBS. Furthermore, the influences of avasimin treatment to the lipid compositions in the liver and the adrenal glands, two key organs for lipid homeostasis and cholesterol-based hormone synthesis, was investigated. Between the PBS-treated and avasimin-treated groups, the SRL images exhibited no difference in the amount of LDs in the liver and the adrenal gland cortex. Also, Raman spectra of LDs were not significantly different between PBS-treated and avasimin-treated groups. Interestingly, the Raman band at 702 cm⁻¹ for the liver tissue was weak, whereas a strong band at 702 cm⁻¹ for the adrenal gland tissue was observed. The results support that the LDs in the liver mainly have triacylglycerol (TG) by hepatic fatty acid de novo synthesis. In contrast, the LDs in the adrenal glands store a large amount of CEs for steroid hormone synthesis. Quantitation of the CE level (FIG. 6B) clearly showed no adverse effects of avasimin treatment to adrenal glands. Together, these results demonstrate the safety of avasimin treatment.

### Example 2

### Material and Methods

Cell Lines and Chemicals:

Immortalized human pancreatic duct epithelial cell line HPDE6 and human pancreatic cancer cell line AsPC-1, BxPC-3, MIA PaCa-2 and PANC-1 were obtained from the American Type Culture Collection (ATCC). All cells were cultured at 37° C. in a humidified incubator with 5% CO₂ supply. Cells were grown in the following media: Keratinocyte Serum Free Medium (KSFM) (Invitrogen) supplemented with 30 μg/ml BPE and 0.2 ng/ml rEGF for HPDE6 cell; DMEM high glucose (Invitrogen) supplemented with 10% FBS for PANC1 cell; RPMI 1640 (Invitrogen) supplemented with 10% FBS for AsPC-1, BxPC-3 and MIA PaCa2 cell. MIA PaCa-2 cell with stable expression of luciferase and mCherry fluorescent protein were obtained from In Vivo Therapeutics Core at Indiana University Simon Cancer Center (Indiana University, IN) and grown in DMEM supplemented with 10% FBS.

Chemicals including cholesteryl oleate, glyceryl trioleate, and simvastatin were purchased from Sigma-Aldrich. Avasimibe used in vitro and in vivo studies were purchased from Selleckchem.com. Human low-density lipoprotein (LDL) was purchased from Creative Laboratory Products (Indianapolis, Ind.) and conjugated with DiI by the authors. Lipoproteindeficient Serum (LPDS) was purchased from Biomedical Technologies (Ward Hill, Mass.).

Human Pancreatic Tissue Specimens:

Frozen specimens of human pancreatic tissues were obtained from Indiana University Simon Cancer Center Solid Tissue Bank. In total 14 pairs of matched normal and cancerous tissues were collected. For each tissue specimen, pairs of adjacent tissue slices were prepared to be used. One slide remained unstained for spectroscopic imaging and the other stained with H&E for pathological examination by a pathologist.

Label-Free Raman Spectromicroscopy:

Label-free Raman spectromicroscopy, including stimulated Raman scattering (SRS) microscopy, coherent anti-stokes Raman scattering (CARS) microscopy and spontaneous Raman spectroscopy, was performed on unstained tissue slices (˜15 μm) or cells without any processing or labeling. SRS imaging was conducted using a femtosecond laser source, with the laser beating frequency tuned to the C-H stretching vibration mode at 2845 cm−1. The typical acquisition time for a 512×512 pixel image was 1.12 second. The power of laser beams were carefully adjusted and no photo-damages were observed to the tissues or cells.

CARS imaging and confocal Raman spectral analysis from individual LDs were performed on a single platform as described previously (40). A 5-picosecond laser at 707 nm was used as excitation beam for Raman spectral acquisition. Acquisition time for a typical spectra from individual LDs was 20 s, with the beam power maintained around 15 mW at sample. For each specimen, at least 10 spectra from individual LDs in different locations or cells were obtained.

In vivo study in orthotopic mouse model: All animal experiments were conducted following protocols approved by Purdue Animal Care and Use Committee (PACUC). NOD/scid/IL2R γ null (NSG) mice were purchased from In Vivo Therapeutics Core at Indiana University Simon Cancer Center (Indiana University, IN) under a Material Transfer Agreement with Jackson Laboratories, Inc. Orthotopic mouse model of pancreatic cancer was established following previously described protocol (Gu D, et al. (2013) Mol Cancer Ther 12(6):1038-1048.). MIA PaCa-2 cells with stable expression of luciferase and mCherry were collected and suspended at a concentration of 10×106 cells/ml. A total of 5×105 tumor cells in 50 μl medium was directly injected into the pancreas of NSG mice at 4-6 weeks old. After recovery from surgery, tumor growth was monitored by bioluminescent imaging using In Vivo Imaging System (IVIS).

In these studies, mice were randomly divided into two groups. For the treatment with avasimibe at 15 mg/kg, intraperitoneal (IP) injection was used on a daily base, starting one week after tumor cell implantation. After treated for 4 weeks, all the mice were sacrificed. Tumors and metastatic lesions in abdominal cavity, lymph nodes, liver, spleen, kidney, lung and heat were visualized with IVIS by mCherry fluorescent signal. Tumor volume and weight were measured ex vivo. Histological examination was performed to tumor and organ tissue slides after stained with Hematoxylin and eosin (H&E).

Statistical Analysis:

One-way ANOVA and Student's t test were used for comparisons between groups. Results were represented as means+/±SDs. Significant differences were considered at *P<0.05, **P<0.01, and ***P<0.001.

Quantitative Analysis of SRS Imaging and Raman Spectra:

LD amount was quantified based on the SRS images using software ImageJ. Due to significantly higher signal level, LDs were picked up by “Threshold” function. Area fraction of LDs out of total area of tissues or cells were measured to quantize the LD amount. CE level in individual LDs was quantified by analyzing the height ratio of the 702 cm−1 peak to 1442 cm−1 peak, which was shown to be linearly proportional to the percentage of CE out of total lipids. The percentage of CE out of total lipids was used to quantize the CE level.

Cell Viability, Migration and Invasion Assay:

Cell viability was measured by Thiazolyl Blue Tetrazolium Blue (MTT) colorimetric assay (Sigma). Cell migration and invasion assays were performed in Transwell chambers (Corning) coated with and without Matrigel (BD Bioscience) respectively. 20% FBS and 0.2 ng/ml EGF were used as chemoattractant. 12 h (migration) or 24 h (invasion) after cells were seeded on top of the membrane, penetrating cells were stained with PI and counted under confocal microscope.

Cell Cycle and Apoptosis Analysis:

PC-3 cells treated with 10 μM avasimibe for 3 days and the untreated ones were collected, fixed, and stained with 50 μg/ml propidium iodide (PI) at 37° C. for 30 min. The DNA content was measured by Cytomics FC500 flow cytometer (Beckman Coulter). Data were processed and analyzed by FlowJo software (Tree Star). Cell-cycle phases and frequencies of each phase were determined by fitting the data with Watson-Pragmatic model. Annexin V/PI staining assay (Life Technologies) was used for detecting apoptotic cells by following manufacturer's protocol.

Fluorescence Imaging of DiI Labeled LDL Uptake:

DiI labeled LDL was made following previously described method (de Smidt P C & van Berkel T J C (1990) Cancer Res 50(23):7476-7482.). Cells were cultured in medium containing 10% lipodeficient serum with or without avasimibe treatment for 2 days. Then DiI-LDL was added to the medium at 50 μg/ml for 3 hours. Fluorescence images were acquired immediately after 3 h incubation using confocal microscope. The LDL uptake was quantified using ImageJ by analyzing the area fraction of uptake LDL particles out of the total cellular area.

Lipid Extraction and Biochemical Assay of Cholesterol:

Lipid extraction from cell pellets and tissues was performed according to Folch et al (Folch J et al. (1957) J Biol Chem 226(1):497-509.). Cells with indicated treatments were collected and counted. CE and free cholesterol were measured according to the manufacturer's protocol (Amplex Red Cholesterol kit from Molecular Probes), and finally normalized by cell number.

Electrospray Ionization Mass Spectrometry (ESI-MS) Measurement of CE:

Lipids were extracted as described above. Cells with indicated treatments were collected and counted. ESI-MS analysis was conducted according to the protocol described previously (Liebisch G, et al. (2006) Biochim Biophys Acta. 1761(1): 121-128.). The relative level of cholesteryl oleate (18:1) was normalized by cell number for comparison between control and avasimibe-treated cells.

Immunoblotting:

After indicated treatments, cells were harvested and lysed in AMI lysis buffer (Active Motif) supplemented with protease and phosphatase inhibitor cocktail. Protein concentration was determined using the Bio-Rad protein assay kit. Protein extraction was subjected to immunoblotting with the antibodies against PTEN (Cell Signaling, 9188S), HMG CoA reductase (Biovision, 3952-100), ACAT-1 (Santa Cruz, sc-69836), GRP78 (Santa Cruz, sc-13968), ATF4 (Cell Signaling, 11815), CHOP (Cell Signaling, 2895), p-ERK1/2 (Cell Signaling, 4370S), ERK1/2 (Cell Signaling, 9102), p-Akt (Cell Signaling, 4060L), Akt (Cell Signaling, 4691), caveolin-1 (Cell Signaling, 3238S), SREBP-1 (Santa Cruz, 13551), and β-actin (Sigma, A5441). β-Actin was used as loading control for normalization.

Immunohistochemistry:

Murine paraffin-embedded slides were deparaffinized and rehydrated. Then antigens were retrieved in antigen unmasking solution (Vector Laboratories) with a 2100-Retriever (PickCell Laboratories). Samples were used to TUNEL assay (Roche, 11684817910) according to manufacturer's protocol. Fluorescent images were taken using Nikon A1R confocal microscopy under 40× objective.

Transfection and gene knock-down: Stable knock-down of ACAT-1 was conducted with ACAT-1 specific shRNA lentiviral transfection (Santa Cruz, sc-29625-V) according to manufacturer's protocol. Scrambled shRNA lentiviral particles (Santa Cruz, sc-108080) was used as control. Transfected cells were selected under 2 μg/ml puromycin treatment for at least 10 days. Transfection of PTEN shRNA plasmid (Santa Cruz, sc-29459-SH) and scramble shRNA plasmid (Santa Cruz, sc-108060) was performed using Lipofectamine® 2000 (Invitrogen, Cat#11668-019) following the manufacturer's protocol. Wild-type pLKO-PTEN and pLKO-GFP plasmid were transfected using the Lipofectamine method. Caveolin-1 plasmid was purchased from Addgene (#14433) and transfected using the Lipofectamine method.

Results:

Aberrant Accumulation of CE in Human Pancreatic Cancer Tissues and Cell Lines, but not in Normal Counterparts:

Using Raman spectromicroscopy, the lipid distribution was mapped and the composition in individual LDs inside single cells was analyzed. Cryo-sections of matched normal and cancerous human pancreatic tissues from same patients were used, of which the pathological statuses were confirmed by a pathologist. Totally 14 pairs of matched normal and cancerous tissues were imaged and analyzed. Our SRS images revealed a much higher level of LD accumulation in cancerous pancreatic tissues than in normal tissues. To make sure that the images and Raman spectra were taken from the cancer cells, but not the stromal cells, histological stained adjacent slides were used to identify the cancer cells. By quantitative analysis of the SRS images, we showed that the amount of LDs in cancer tissues is over 20 times higher than that in normal tissues.

To assess the composition of LDs in cancer and normal tissues, Raman spectra from individual LDs inside the cells were acquired and analyzed. The LDs in pancreatic cancer cells contained high level of CE, indicated by the characteristic ester bond vibration mode at 1742 cm⁻¹ and the cholesterol ring vibration mode at 702 cm⁻¹. Multiple Raman spectra from individual LDs in the same cells were recorded to ensure consistency. It is worth noting that spectra from normal tissues showed no peak at 702 cm⁻¹ but a strong peak at 2930

cm⁻¹, indicating high protein content in those LDs. Using emulsions mixed by cholesteryl oleate and glyceryl trioleate, it was verified that height ratio of the Raman peak at 702 cm⁻¹ to the peak at 1442 cm⁻¹ (CH2 bending vibration mode) is linearly proportional to the molar percentage of CE. Based on this calibration curve, it was found that the percentage of CE in pancreatic cancer tissues ranged from 60% to 95%, while the percentage in normal tissues was within 10-20%. The lipids in human pancreatic cell lines were further studied, including normal immortalized pancreatic epithelial cell line HPDE6, pancreatic cancer cell lines MIA PaCa-2, PANC-1, AsPC-1 and BxPC-3. Consistent with the human specimen data, SRS imaging and Raman spectral analysis revealed higher CE levels in cancer cells than in normal cells. Collectively, these data suggest that CE accumulation is a metabolic event that occurs primarily in pancreatic cancer cells but not in normal cells.

CE Accumulation in Pancreatic Cancer is Regulated by PTEN Activity and Mediated by Both De Novo Cholesterol Synthesis and LDL Uptake:

Although all pancreatic cancer cell lines have higher CE levels than normal cell line, it was found that CE levels varied in different cancer cell lines. Specifically, MIA PaCa-2 and PANC-1 cells had much higher levels of CE than AsPC-1 and BxPC-3 cells. Since PTEN loss has been shown to drive the CE accumulation in prostate cancer, it was asked if CE level variation in pancreatic cancer cell lines is due to different PTEN expression level. To confirm this, immunoblotting analysis of PTEN expression was performed, and it was found that the PTEN protein levels were negatively correlated with the CE level in pancreatic cancer cells. To determine whether PTEN regulates CE accumulation, knock-down of PTEN by specific shRNA in AsPC-1 cells was conducted and induced overexpression of wild-type PTEN in MIA PaCa-2 cells. As expected, knock-down of PTEN by shRNA significantly increased CE levels in AsPC-1 cells, while overexpression of wild-type PTEN significantly reduced CE in MIA PaCa-2 cells. These data collectively demonstrated that CE accumulation in pancreatic cancer is regulated by PTEN activity.

Cancer cells obtain cholesterol either from de-novo synthesis or by uptake from extracellular nutrition via LDL. To investigate which pathway contributes to CE accumulation in pancreatic cancer, simvastatin, a specific inhibitor of 3-hydroxy-3-methylglutaryl coenzyme A (HMG-CoA) reductase, the rate-limiting enzyme in the cholesterol synthesis pathway, was used to block the de novo synthesis pathway. For the alternative pathway, lipodeficient-serum was applied to deplete the extracellular cholesterol carried in LDLs. Either lipodeficientserum supplementation or HMG-CoA reductase inhibition significantly reduced the CE level, indicating that both de novo synthesis and LDL uptake pathways contribute to CE accumulation in pancreatic cancer cells.

After cellular uptake, LDL-delivered CE is hydrolyzed into free cholesterol. The excess free cholesterol is then re-esterified by acyl coenzyme A: cholesterol acyltransferase (ACAT)-1 or ACAT-2, of which the latter one is majorly expressed in intestinal mucosal cells in human. Consistent with the increased CE level, an overexpression of ACAT-1 and HMGCoA reductase in human pancreatic cancer tissues was observed. Inhibition of ACAT-1 by either a potent inhibitor, avasimibe, or specific shRNA knock-down effectively removed CE in cancer cells (FIG. 8). Reduction of CE levels by avasimibe was confirmed by electrospray ionization mass spectrometry (ESI-MS) analysis, which also revealed that the principal form of CE in pancreatic cancer cells was cholesteryl oleate. Avasimibe treatment also significantly reduced uptake of LDL as shown by fluorescence imaging and quantitative analysis of intracellular DiI labeled LDL, indicating that LDL uptake is a tightly controlled process in response to the regulation of cholesterol hemostasis. Together, the results show that CE accumulation in pancreatic cancer arises from both de novo synthesis and LDL uptake, and is mediated by the ACAT-1 enzyme.

Blocking Cholesterol Esterification Suppresses Pancreatic Cancer Growth and Metastasis:

Considering CE accumulation is a cancer specific event, it was further tested whether the cholesterol esterification process could be a potential target for cancer therapy. By using avasimibe, a potent inhibitor of ACAT-1, it was found that pancreatic cancer cells MIA PaCa-2 and PANC-1 were more sensitive to ACAT-1 inhibition than normal HPDE6 cells. The IC50 of avasimibe for MIA PaCa-2, PANC-1, and HPDE6 are 11.03 μM, 23.58 μM, and 52.81 μM, respectively. Inhibition of ACAT-1 by avasimibe at 10 μM significantly reduced the proliferation rate of MIA PaCa-2 and PANC-1 cells. To confirm that the anti-cancer effect of avasimibe is specific to ACAT-1 inhibition, knock-down of ACAT-1 by specific shRNA was performed. As predicted, MIA PaCa-2 cells with ACAT-1 knock-down showed a much reduced proliferation rate. Moreover, using the transwell method cell migration and invasion assays were performed. Inhibition of ACAT-1, either by avasimibe or shRNA knock-down, significantly reduced MIA PaCa-2 cell migration and invasion rates. Together, these results show that cancer cells are more sensitive to blockage of cholesterol esterification, leading to a decrease in pancreatic cancer cell migration and invasiveness.

To explore the anti-cancer effect of ACAT-1 inhibition in vivo, a well-established orthotopic mouse model of pancreatic cancer was used. MIA PaCa-2 cells with luciferase and mCherry expression were orthotopically injected into the pancreas. Tumor growth was monitored weekly by imaging the luminescence signal in vivo. Firstly, MIA PaCa-2luc/mCherry cells with or without stable knock-down of ACAT-1 were used. Compared to the ACAT-1 wild-type group, the ACAT-1 knockdown group developed significantly smaller tumors after 5 weeks of tumor cell implantation. Tumor growth was dramatically suppressed as indicated by the luciferase activity from the cancer cells, while no obvious loss in body weight was observed. Ex vivo measurement of the tumor volume and weight confirmed the suppressive effect on tumor growth by ACAT-1 knock-down. Metastatic lesions in lymph nodes and distant organs (e.g., liver, spleen and lung) were assessed by imaging at the endpoint of the study. As expected, metastatic lesions were readily detected in ACAT-1 wild-type group while no or very few lesions were detectable in the ACAT-1 knock-down group. Quantification of the number of metastatic lesions exhibited significant differences between these two groups. Reduction of CE level in the tumor tissues confirmed the effect was due to inhibition of ACAT-1.

To further explore the potential of ACAT-1 inhibition for pancreatic cancer treatment, avasimibe was administered intraperitoneally to the orthotopic model at a dosage of 15 mg/kg per day (Yue S, et al. (2014) Cell Metab. 19(3):393-406.). Avasimibe treatment for 4 weeks significantly suppressed tumor size, tumor growth rate, tumor volume and weight. Metastatic lesions in lymph nodes and distant organs were largely suppressed. Importantly, avasimibe did not induce body weight loss. Pathological assessment also confirmed that no obvious toxicity was induced to other organs (e.g., liver, kidney, lung and spleen), as shown by hematoxylin and eosin (H&E) staining. Reduction of CE amount was also confirmed with SRS imaging and Raman spectral analysis. These data collectively demonstrated the potential of using ACAT-1 inhibition for pancreatic cancer therapy.

Inhibition of ACAT-1 Induces ER Stress and Apoptosis in Pancreatic Cancer:

To understand the molecular mechanisms by which ACAT-1 inhibition lead to less aggressive tumor growth and metastases, the downstream effects were examined. The ACAT-1 enzyme esterifies free cholesterol to its esterified form, which can be stored in the LDs for maintenance of cholesterol homeostasis. It was proposed that cholesterol esterification may provide a way to minimize the cytotoxicity of excess free cholesterol caused by increased de novo cholesterol synthesis and LDL uptake. As anticipated, free cholesterol levels gradually increased with avasimibe treatment from low to high concentration. Increased free cholesterol was also detected in the mouse pancreatic tumor tissues treated with avasimibe.

Increased intracellular free cholesterol levels have been previously reported to be cytotoxic in macrophages due to increased ER stress and subsequent apoptosis (Warner G J et al. (1995) J Biol Chem 270(11):5772-5778.). To test the effect of avasimibe on ER stress in pancreatic cancer cells, several ER stress markers were used, including 78 kDa glucose-regulated protein (GRP78), activating transcription factor 4 (ATF4) and C/EBP homologous protein (CHOP). By immunoblotting, it was shown that GRP78 expression level gradually increased over time after avasimibe treatment, indicating the release of ER chaperone GRP78. Release of GRP78 activated subsequent unfolded protein response pathway, leading to an increase of transcription factor ATF4 within 12 hours after treatment. ATF4 further induced expression of pro-apoptotic factor CHOP, expression of which appeared after 12 hour treatment and increased from 12 to 48 hour. ER stress in MIA PaCa-2 cells treated with avasimibe from low to high concentrations was further confirmed, as indicated by a gradual increase of GRP78. An increase of GRP78 expression was also observed in MIA PaCa-2 cells with ACAT-1 knock-down.

ACAT-1 inhibition induced ER stress eventually led to apoptosis of cancer cells. Using annexin V/propidium iodide staining and cell cycle analysis by flow cytometry, it was shown that the number of apoptotic cells largely increased in avasimibe treated MIA PaCa-2 cells.

Increased apoptotic cells were also observed in avasimibe treated tumor tissues, which were detected by TUNNEL assays. Taken together, it was concluded that ACAT-1 inhibition induced an increase of intracellular free cholesterol, ER stress and apoptosis in pancreatic cancer cells.

Inhibition of ACAT-1 Downregulates MAPK Activity Mediated by Caveolin-1:

Cholesterol plays essential roles in maintaining membrane structure and function, like lipid rafts, through which a number of cellular signaling pathways (e.g., caveolin-1 signaling) are transduced.

Caveolin-1 as a regulator of cellular cholesterol homeostasis is considered a marker for pancreatic cancer progression. Particularly, a promoting role of caveolin-1 on pancreatic cancer metastasis has been reported. In this study, a reduction of caveolin-1 expression levels was observed after ACAT-1 inhibition, as well as expression of SREBP-1, caveolin-1, ERK1/2 and p-ERK1/2. Reduced expression of caveolin-1 and p-ERK1/2 were confirmed by knock-down of ACAT-1. The effect on caveolin-1 is probably mediated by SREBP-1, which senses the intracellular cholesterol homeostasis. Meanwhile, caveolin-1 may play an important role in mediating the action of SREBP1 on MAPK pathways, which are known to play essential roles in cancer cell metastasis. It was further shown that knockdown of caveolin-1 significantly reduced the expression of p-ERK1/2. Overexpression of caveolin-1 in MIA PaCa-2 cells rescued the reduced cell migration and invasion caused by avasimibe treatment. Together, the data suggest that downregulation of caveolin-1 and MAPK signaling pathways contribute to the reduction of cancer proliferation and metastasis following ACAT-1 inhibition.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

What is claimed is:

1. A formulation comprising:
an original ACAT-1 inhibitor; and
human serum albumin, wherein the combination of the original ACAT-1 inhibitor and human serum albumin decreases the hydrophobicity of the original ACAT-1 inhibitor.

2. The formulation of claim 1 wherein the original ACAT-1 inhibitor has a loading of from about 10 wt % to about 30 wt % with respect to human serum albumin.

3. The formulation of claim 1 wherein the original ACAT-1 inhibitor has a loading of from about 15 wt % to about 25 wt % with respect to human serum albumin.

4. The formulation of claim 1, wherein the original ACAT-1 inhibitor is avasimibe.

5. The formulation of claim 4 wherein the original ACAT-1 inhibitor has a loading of from about 10 wt % to about 30 wt % with respect to human serum albumin.

6. The formulation of claim 4 wherein the original ACAT-1 inhibitor has a loading of from about 15 wt % to about 25 wt % with respect to human serum albumin.

7. A method of treating a mammal having a disease or condition that would benefit from inhibiting ACAT-1 comprising administering a therapeutically effective amount of the formulation of claim 1.

8. A method of claim 7 wherein the original ACAT-1 inhibitor is avasimibe.

9. A method of treating a mammal having a disease or condition that would benefit from inhibiting ACAT-1 comprising administering a therapeutically effective amount of a formulation comprising:
an original ACAT-1 inhibitor; and
human serum albumin, wherein the combination of the original ACAT-1 inhibitor and human serum albumin decreases the hydrophobicity of the original ACAT-1 inhibitor.

10. The method of claim 9 wherein the original ACAT-1 inhibitor has a loading of from about 10 wt % to about 30 wt % with respect to human serum albumin.

11. The method of claim 9 wherein the original ACAT-1 inhibitor has a loading of from about 15 wt % to about 25 wt % with respect to human serum albumin.

12. The method of claim 9 wherein the original ACAT-1 inhibitor is avasimibe.

13. The method of claim 9 wherein the disease is cancer.

14. The method of claim 13 wherein the cancer is prostate, pancreatic, lung or colon cancer.

15. The method of claim 13 wherein the cancer is pancreatic cancer.

16. The method of claim 9 wherein the formulation is administered systemic or oral administration.
